(19) United States
(12) Patent Application Publication
(10) Pub. No.: US 2010/0210600 A1
(43) Pub. Date: Aug. 19, 2010

(54) Methods and Compositions for Treating Urogenital Disorders

(75) Inventors: Alan E. KLIGERMAN, Egg Harbor Township, NJ (US); Joseph V. DITROLIO, Roseland, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Egg Harbor Township, NJ (US)

(21) Appl. No.: 12/708,083

(22) Filed: Feb. 18, 2010

Related U.S. Application Data
(60) Provisional application No. 61/153,719, filed on Feb. 19, 2009.

(51) Int. Cl. A61K 31/661; A61M 31/00; A61M 25/00; A61K 9/02; A61M 29/00; A61F 2/04; A61B 1/307; A61P 15/00

(52) U.S. Cl. 514/129; 604/514; 604/523; 604/288; 604/96.01; 623/23.7; 600/135

(57) ABSTRACT

Glycerophosphate salts have been found to drastically improve the treatment of urogenital disorders such as urethral strictures and vaginal atrophy. Methods, devices and kits are described for treating urogenital disorders using a composition comprising an effective amount of a glycerophosphate salt.

### CROSS-REFERENCE TO RELATED APPLICATIONS

This application is entitled to and claims the benefit of the priority pursuant to 35 U.S.C. §119(e) of U.S. Provisional Patent Application No. 61/153,719, filed Feb. 19, 2009, the disclosure of which is hereby incorporated by reference in its entirety.

### BACKGROUND OF THE INVENTION

Urogenital disorders can be irritating, painful and difficult to cure. For example, urethral strictures, a common clinical condition, characterized by the narrowing of the urethra, often by a noncompliant section of scar tissue, are both pathological and potentially clinically damaging. Urethral strictures are generally caused by either injury-related trauma to the urinary tract or by a viral or bacterial infection of the tract. The body's attempt to repair the damage caused by the injury or infection creates a buildup of scar tissue in the tract resulting in a significant narrowing or even closure of the passage. The scarring tends to recur year after year, spontaneously. Instrumentation of the urethra, particularly before the advent of flexible uro-endoscopy, was (and remains) an important causative event. The obstruction of the urethra by the stricture leads to symptoms such as hesitation for urination, decreased force of urinary stream, urinary terminal dribbling, increased frequency of urination, pain during urination, inability to fully empty the bladder, in certain cases the complete inability to urinate, which is a medical emergency. In addition, such obstruction may lead to damage to the urinary bladder, ureters and kidneys.

Urinary strictures are generally difficult to cure or even ameliorate. Urinary strictures can be dense, rigid, and resistant to stretching. They are localized mostly in the membranous or bulbous parts of the urethra. A common non-surgical method for treating urethral strictures is dilation of the urethra, for example by probing the strictures with elongated members or probes to enlarge them. The insertion of a conventional dilation member, such as a bougie or a stent in urethra, particularly in males, is a very complicated and painful operation, requiring high skill and concentration. Complications such as bleeding, perforations of the urethral wall and other injuries, urinary fever, prostatitis, epididymitis, and the like may occur. As a result of the dilation, additional scar tissues may be produced, which in turn could cause luminal obstruction and treatment failure.

The surgical method for treating urethral strictures, such as by internal urethrotomy or urethroplasty, involves incising the strictures. However, the surgery is complicated and may result in complications, such as recurrence of strictures, bleeding, extravasation of irrigation fluid into perispongial tissues and increasing fibrotic response, etc.

Vaginal atrophy, another example of urogenital disorders, is commonly experienced by a large number of postmenopausal women. It is caused by an inflammation of the vagina and/or the outer urinary tract due to the thinning and shrinking of the tissues and decreased lubrication, often as a result of a lack of the reproductive hormone, e.g., estrogen. Vaginal atrophy includes genital symptoms, such as dryness, itching, burning, soreness, pressure, white discharge, malodorous discharge due to infection, painful sexual intercourse, bleeding after intercourse, and occurrence of sores and cracks in the vagina. Vaginal atrophy also includes urinary symptoms, such as painful urination, blood in the urine, increased frequency of urination, incontinence, and increased likelihood and occurrence of infections. The shrinkage of the tissues can be extreme enough to make intercourse impossible.

Estrogen therapy has been used to treat vaginal atrophy. The doctor can prescribe an estrogen pill, a topical estrogen cream, suppositories to insert in the vagina, an estrogen skin patch or a vaginal estrogen ring. Estrogen cream or suppositories are inserted into the vagina using an applicator. An estrogen patch is applied to deliver estrogen through the skin. A vaginal ring is placed in the vagina and slowly releases estrogen directly to the vaginal tissues. However, the use of estrogen, alone or in combination with progesterone, has been questioned because of potentially increased risk of heart attack and stroke in women taking the estrogen therapy.

Water-soluble lubricants do not contain estrogen, which can be purchased without prescription at drug stores, pharmacies or grocery stores, can be used to as needed to relieve vaginal dryness and moisten tissue. However, the currently available lubricants are often only helpful in mild cases.

Therefore, there is a continuing need for an improved and relatively inexpensive means for treating urogenital disorders, such as urethral strictures and vaginal atrophy. Preferably, such a means is non-toxic, non-hazardous and without significant side effects.

### BRIEF SUMMARY OF THE INVENTION

It is now discovered that administering a glycerophosphate salt to the urethra of a subject unexpectedly results in drastic improvement in the treatment of urethral strictures.

In one general aspect, an embodiment of the present invention relates to a method of treating a urogenital disorder in a subject. The method comprises topically administering an effective amount of a glycerophosphate salt to an organ of the urogenital system of the subject, wherein the organ suffers from the urogenital disorder.

In another general aspect, an embodiment of the present invention relates to a device for treating a urogenital disorder in a subject. The device comprises an applicator associated with an effective amount of a glycerophosphate salt, wherein the applicator is adapted for placement into an organ of the urogenital system of the subject. In yet another general aspect, an embodiment of the present invention relates to a kit for treating a urogenital disorder in a subject. The kit comprises:

(1) a composition comprising an effective amount of a glycerophosphate salt; and

(2) instructions for administering the composition to an organ of the urogenital system of the subject.

In another general aspect, an embodiment of the present invention relates to a method of diagnosing a urogenital disorder in a subject. The method comprises:
a. associating an effective amount of a glycerophosphate salt with an endoscope;
b. inserting the endoscope associated with the glycerophosphate salt into the urogenital system of the subject; and
c. detecting the urogenital disorder using the endoscope.

In a preferred embodiment, the glycerophosphate salt is calcium glycerophosphate (CGP).

In particular embodiments, the urogenital disorder is a urethral stricture or vaginal atrophy.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

As used herein, the term “subject” refers to an animal, preferably a mammal, who/which has been the object of treatment, observation or experiment. Examples of a subject can be a human, a livestock animal (beef and dairy cattle, sheep, poultry, swine, etc.), or a companion animal (dog, cat, horse, etc).

As used herein, the term “instructions” when used in the context of a kit includes a publication, a recording, a diagram or any other medium of expression which can be used to communicate the usefulness of the kit for its designated use. The instructions can, for example, be affixed to or included within a container for the kit.

As used herein, a “urogenital disorder” includes a disease, disorder, or condition in the urogenital system, whether the disorder resulting from an injury or a disease in the urogenital system and whether the disorder is temporary or chronic.

As used herein, an “organ of the urogenital system” includes any of the sex organs, the urinary system organs, and the excretion system organs of the subject. In embodiments of the present invention, the “organ of the urogenital system” is the urethra, vulva, vagina, cervix, outer urinary tract, or rectum of the subject.

In one embodiment of the present invention, the urogenital disorder is a urogenital disorder, which includes, but is not limited to, urethritis, urethral abscess, urethral stricture, urethral fistula, urethral diverticulum, urethral trauma, sympathetic inflammation and urethral caruncle.

As used herein, the term “urethral stricture” refers to a narrowing of the urethra in a subject and any disease, disorder or condition the subject suffers as a result of the narrowing. The urethral stricture can be caused by any reasons, including but not limited to, an injury or a disease to the urethra. In one embodiment, the urethral stricture arises after a surgical procedure to the subject, such as a catheterization, a urethral dilatation, an internal urethrotomy, or a urethroplasty of the urethra of the subject. In another embodiment, the urethral stricture arises after an infection of the urethra of the subject, such as an infection by a fungus, protozoa, yeast, bacterium, or virus. In yet another embodiment, the urethral stricture arises after a non-surgical trauma at the urethra site.

The urethral stricture can occur anywhere in the urethra. In one embodiment, the urethral stricture is an anterior urethral stricture that can be secondary to scarring in the spongy erectile tissue of the corpus spongiosum. In another embodiment, the urethral stricture is a posterior urethral stricture that can be a result of fibrotic process that narrows the bladder neck and usually results from a distraction injury secondary to trauma or surgery, such as radical prostatectomy. In yet another embodiment, the urethral stricture can be formed in the bulbar urethra between the posterior and anterior urethral segments, not usually noticeable until later in life, as it fails to permit a larger urine flow as the balance of the urethra does with growth; thus, it only impedes urinary flow relative to the rest of the urethra after puberty.

In another embodiment of the present invention, the urogenital disorder is a genital disorder, which includes, but is not limited to, vaginitis, vaginal atrophy, wounds, or vaginal trauma. The vaginitis can be radiation vaginitis or chemical vaginitis. The wounds can be surgical wounds or trauma wounds.

As used herein, “vaginal atrophy” refers to thinning of the membranes of the vulva, the vagina, the cervix, and also the outer urinary tract, along with considerable shrinking and loss in elasticity of all of the outer and inner genital areas, and any disease, disorder or condition the subject suffers as a result of the thinning, the shrinking and the loss in elasticity. The vaginal atrophy can be caused by the decrease in estrogen, which can be a natural result of menopause. Other causes of decreased estrogen levels are decreased ovarian functioning due to radiation therapy or chemotherapy, immune disorder, removal of the ovaries, after pregnancy, during lactation, idiopathic, and because of the effects of the use of various medications such as (Tamoxifen (Nolvadex®), Danazol (Danocrine®), Medroxyprogesterone (Provera®), Leuprolide (Lupron®), Nafarelin (Synarel®).

In yet another embodiment of the present invention, the urogenital disorder is a rectal disorder, which includes, but is not limited to, constipation, bleeding, hemorrhoids, proctalgia, rectal prolapse, radiation proctitis, rectal fissure, rectal stricture, etc.

The term “effective amount” as used herein means that amount of a glycerophosphate salt that accelerates the treatment of a urogenital disorder and/or reduces one or more symptoms related to the urogenital disorder, in a subject as compared to an otherwise identical treatment without the effective amount of glycerophosphate salt. Symptoms related to the urogenital disorder include, but are not limited to, inflammation, pain, recurrent scarring, and increased likelihood and occurrence of infections to the urogenital system; urinary symptoms, such as the hesitation for urination, decreased force of urinary stream, urinary terminal dribbling, involuntary loss of urine, increased frequency of urination, pain during urination, blood in the urine, inability to fully empty the bladder, complete inability to urinate; genital symptoms, such as dryness, itching, burning, soreness, pressure, white discharge, malodorous discharge due to infection, in painful sexual intercourse, bleeding after intercourse, and occurrence of sores and cracks in the vagina; and rectal symptoms, such as constipation, bleeding, hemorrhoids, proctalgia, rectal prolapse, radiation proctitis, rectal fissure, rectal stricture, etc.

In an embodiment of the present invention, the application of an effective amount of a glycerophosphate salt to the urethra of a subject results in a clinically observable beneficial effect in the reduction of one or more symptoms related to the urogenital disorder, such that the symptoms are about 90%, 80%, 70%, 60%, 50%, 40%, 30%, 20%, 10% or less, than the symptoms that would have resulted from the urogenital disorder had the subject not received an effective amount of the glycerophosphate salt.

In view of the present disclosure, standard procedures can be performed to evaluate the effect of the administration of a glycerophosphate salt to a subject, thus allowing a skilled artisan to determine the effective amount of the glycerophosphate salt to be administered to the subject. For example, the clinically observable beneficial effect of a glycerophosphate salt in treating urethral strictures can be observed by directly measuring the urethral caliber after administration of the glycerophosphate salt to the urethra. The effect can also be observed by monitoring the volume and frequency of urination of the subject.

The clinically observable beneficial effect can be a situation that symptoms related to a urogenital disorder are prevented from further development or aggravation, or develop to a lesser degree than without administration of the composition of the present invention, when a composition of the present invention is administered to a subject after the symptoms are observable. The clinically observable beneficial effect can also be a situation in which symptoms related to a urogenital disorder are prevented from occurring or subsequently occur to a lesser degree than without administration of the composition of the present invention, when a composition of the present invention is administered to a subject before the symptoms are observable.

One skilled in the art will recognize that the “effective amount” of a glycerophosphate salt to be used in the instant invention can vary with factors, such as the particular subject, e.g., age, diet, health, etc., size of the wound, severity and complications of the urogenital disorder sought to be treated, the mode of administration of a glycerophosphate salt, the particular glycerophosphate salt used, the time when the glycerophosphate salt is first applied to the wound, etc. Standard procedures can be performed to evaluate the effectiveness of the administration of a glycerophosphate salt to a urogenital disorder, thus allowing a skilled artisan to determine the effective amount of the glycerophosphate salt to be administered to an organ of the urogenital system, such as the urethra, the vagina or the rectum, in view of the present disclosure.

For topical administration, the glycerophosphate salt can be dissolved and suspended in a cream or a gel. The effective amount of a glycerophosphate salt per administration to the urogenital system can be, for example, about 0.1 gram to about 15 grams, and preferably about 3.5 grams to about 10 grams per 100 grams of cream or gel. The preferred daily dosage of a glycerophosphate salt administered to the urogenital system can be about 3.5 grams to about 10 grams, and more preferably about 5.0 grams to about 8.5 grams per 100 grams of cream. However, the number of doses per day and the quantity of the glycerophosphate salt which can be administered to a subject can be almost unlimited, with the limiting factor being only that point at which the glycerophosphate salt itself reaches a level at which it either irritates the organ of the urogenital system from its inherent anhydrous grit characteristics, or it will not flow, or both. Even if the CGP is “micronized”, i.e., reduced in size by milling or other means to an almost flour-like particle size, the potential for grit-induced irritation can still be present for this hypersensitive situation.

As used herein, the term “glycerophosphate salt” refers to a chemical compound that is derived from glycerophosphate, in which one or more of the hydrogens of the phosphate group of glycerophosphate are replaced by a basic radical, in particular embodiments by a metal ion. As used herein, the term “glycerophosphate” refers to an anion of a phosphoric ester of glycerol, in which a carbon atom of glycerol bonds to an oxygen atom in the phosphate group of phosphoric acid. A glycerophosphate salt can be a chiral molecule, i.e., it can exist in two forms that are nonsuperimposable mirror images. It is intended that the present invention includes within its scope the stereochemically pure isomeric forms of a glycerophosphate salt and/or their racemates.

In particular embodiments, methods of the invention utilize one or more glycerophosphate salts selected from the group consisting of calcium glycerophosphate (CGP), magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, and glycerophosphate strontium.

The effective amount of a glycerophosphate salt takes into account of the efficacy of the particular glycerophosphate salt used. For example, extreme caution is taken to use lithium glycerophosphate in a method according to an embodiment of the present invention, because its effects on mood and its possible adverse effects on cardiac and renal function. The effective amount also takes into account of other properties of the glycerophosphate salt, such as toxicity (e.g., cupric glycerophosphate).

In a preferred embodiment, the invention relates to the use of calcium glycerophosphate. As used herein, the term “calcium glycerophosphate” or “CGP,” also known as “glycerophosphate calcium,” refers to a chemical compound having a molecular formula of C₃H₇CaO₆P in its anhydrous form. “CGP” can also exist as a hydrate, including the monohydrate and the dihydrate. Examples of calcium glycerophosphate include, but are not limited to, any one, or any combination of two or more of the three isomers of CGP, namely β-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca).

Calcium glycerophosphate available from various commercial sources can be used in the present invention. In one embodiment, Cellerity®, a topical lotion available from AkPharma Inc. (Pleasantville, N.J. 08232), can be used in the present invention. Other commercially available CGP also includes CGP available from Astha Laboratories Pvt, Ltd, B-4, Industrial Estate, Sanathnagar, Hyderabad-18, India, and Seppic Inc., 30 Two Bridges Road, Fairfield, N.J.

In another embodiment, the invention utilizes a glycerophosphate salt other than calcium glycerophosphate. Calcium glycerophosphate may be contraindicated for persons with poor renal function or who are in renal failure. The presence of calcium ion may also suppress activities of certain drugs, e.g., certain antibiotic drugs, that are co-administered with the glycerophosphate salt. Therefore, methods of the invention also utilize one or more glycerophosphate salts selected from the group consisting a Na, K, Mg, Ag, or Sr salt of glycerophosphate, or any other non-calcium glycerophosphate salts described herein or known to a person skilled in the art.

Embodiments of the present invention comprise a method, a device and a kit for treatment of a urogenital disorder, particularly a urethral stricture or vaginal atrophy.

The use of glycerophosphate salts, such as calcium glycerophosphate, has been disclosed in various patent applications for purposes of skin care, wound healing and scar minimization. While not wishing to be bound by theory, it is believed that some of the advantages of calcium glycerophosphate are at least due to the unique joint effects of both the calcium ion and the glycerophosphate ions in the molecule. The calcium ion and the glycerophosphate ion are demonstrated to be of importance in cellular repair and repair of tissue damage via a number of mechanisms explained in literature and patents granted and pending, see for example, US20040037766. In addition, glycerophosphate ion is believed to be an agonist for the longevity of the signaling biolipid, sphingosine-1-phosphate (S1P), which is formed by phosphorylation of the cellular biolipid sphingosine in nano-quantities. S1P in turn is an active agent in cellular repair in a number of respects.

It is now discovered that topically administering a glycerophosphate salt to the urogenital system, such as the urethra, drastically improved the treatment and outcome of a urogenital disorder, such as a urethral stricture, in patients where other interventions have been unsuccessful.

In one general aspect, the present invention relates to a method of topically administering a glycerophosphate salt to an organ of the urogenital system of a subject, for the purposes of treating a urogenital disorder in the organ, whether the urogenital disorder is temporary or chronic, or is endogenous, traumatic or iatrogenic in origin.

In one embodiment of the present invention, the effective amount of the glycerophosphate salt is directly topically administered to the organ, such as the urethra, the vagina or the rectum, preferably at or near the site of the urogenital disorder. Any conventional methods of administering a compound topically to the urogenital system of a subject can be used in the present invention in view of the present disclosure.

In a particular embodiment of the present invention, for directly topical administration to the urogenital system, the effective amount of a glycerophosphate salt is first associated with an applicator, for example, by coating the surface of the applicator or impregnate the applicator. The applicator associated with the glycerophosphate salt is then placed into an organ of the urogenital system of the subject, and the glycerophosphate salt is released from the applicator and administered topically to the organ. The applicator can be any member of a device that is adapted for insertion into the urogenital system of a subject. Examples of the applicators include, but are not limited to, a solid rod, a catheter, a stent, a dilator, a suppository, or a sound.

In another embodiment of the present invention, the effective amount of a glycerophosphate salt is administered to the urogenital system through a hollow instrument, such as a catheter or a stent, which is already placed in the urogenital system. The composition can be administered by injections, instillations, permeations, diffusions, infusions, etc. via pressure or gravity through semi-permeable membranes on the catheter. The catheter can be permanently or semi-permanently placed in an organ of the urogenital system, such as the urethra, vagina or rectum. For example, in a treatment for a urethral stricture, an effective amount of a glycerophosphate salt can be administered to the urethra via a semi-permanent (e.g., 2 hrs.) urethral stent that inhibits voiding during treatment, thus preventing the washout of the glycerophosphate salt for that period. In another embodiment, compositions in an indwelling catheter can self-administer superficially to a catheter already placed in an organ of the urogenital system via catheter manipulation to rebathe the endothelium or surface of the organ.

In another embodiment of the present invention, the effective amount of a glycerophosphate salt is administered to the urogenital system by a procedure such as enema, or lavage.

For the purpose of laving an area in the urogenital system, the formulation can be a less-viscous fluid of the glycerophosphate salt, which can be made using methods known to those skilled in the art in view of the present disclosure.

In an embodiment of the present invention, an effective amount of a glycerophosphate salt is topically administered to the urogenital system of a subject in combination with one or more other methods of treatment for urogenital disorders. The effective amount of the glycerophosphate salt can be administered to the urogenital system before, after, or simultaneously with the one or more other treatment methods for the urogenital disorders.

According to an embodiment of the present application, in a method of treatment for a urogenital disorder related to infection or inflammation of the urogenital system, such as urethritis, urethral abscess, vaginitis, the effective amount of a glycerophosphate salt is topically applied to the urogenital system in combination with one or more medications conventionally used for treating such disorders. The medications include, but are not limited to, clotrimazole (Mycelex®), doxycycline (Vibramycin®), fluconazole (Diflucan®), metronidazole (Flagyl®), nitrofurantoin, nystatin (Mycostatin®), clindamycin, Betadine®, hexaclorophine (Phisohex®), or a combination of sulfamethoxazole and trimethoprim (Septrin, Bactrim).

Because calcium binds to most or all antibiotics, the effective amount of a glycerophosphate salt is preferably administered to the urogenital system before or after the administration of an antibiotic. Because mycins are very irritative, the application of a glycerophosphate salt, such as CGP, at an appropriate time before or after topical administration of a mycin can minimize mycin-caused irritation.

In a preferred embodiment of the present invention, the urogenital disorder is a urethral stricture. The effective amount of glycerophosphate salt can be administered to the urethra alone or in combination with one or more conventional methods for treating urethral strictures. The conventional methods can be selected from the group consisting of dilation, catheterization, internal urethrotomy and urethroplasty, and in-dwelling stents. The use of a glycerophosphate salt, such as calcium glycerophosphate, can minimize and/or heal damage to the urethra when the urethra is surgically invaded by sounds, insertion instruments, stents, etc., during the conventional treatment. A glycerophosphate salt can be administered to the urethra before, after or simultaneously with the conventional treatment.

Urethral strictures can result from the scar formation on an insulted or disrupted delicate urethral surface. Surgical excision of an old scab or other fibroblastic materials has been used to treat strictures. For example, it has been suggested that strictures situated in the bulbar urethra are best treated by excision and end-to-end anastomosis if they are short enough, or by patch urethroplasty using a buccal mucosal graft if they are longer; penile strictures due to lichen sclerosus often require a staged approach to reconstruction, again using buccal mucosal grafts; pelvic fracture urethral injury is best treated by bulbo-prostatic anastomotic urethroplasty. See Andrich and Mundy, European Urology, 2008, 54:1031-1041. However, new scarring from the surgical excision can cause recurrence of strictures.

As it was described in U.S. Patent Application No. 61/133,687, glycerophosphate salts have been found to hasten the healing of wounds and minimize the formation of scars, particularly when it is first applied to a wound about 0 to about 7 days after the wound is inflicted. Without wishing to be bound by theory, it is believed that administering a glycerophosphate salt to the urogenital system would at least minimize scar formation on an insulted or disrupted delicate urogenital surface, thus reduce urethral strictures resulting from the scar formation. The glycerophosphate salt would also act as an acid neutralizer in the urethra to reduce pain that would otherwise be caused by normal acidity of the urine on the injured urethral surface. The glycerophosphate salt also heals damaged cells in the urethra and maintains cellular health of the normal or healed urethral cells.

Accordingly, an embodiment of the present invention relates to a method of treating a urogenital disorder in a subject, which further comprises a surgical treatment of the urogenital disorder in the organ, wherein the effective amount of the glycerophosphate salt is first topically administered to the organ about 0 to about 7 days after the surgical treatment.

In a preferred embodiment, the urogenital disorder is a urethral stricture.

In another embodiment of the present invention, the urogenital disorder is vaginal atrophy. The effective amount of a glycerophosphate salt can be administered to the vulva, the vagina, the cervix, or the outer urinary tract of the subject, for example, as vaginal lubricant/sexual lubricant. Unlike estrogen, calcium glycerophosphate is non-toxic, non-hazardous and has no known side effects. Therefore, CGP is particularly desirable for a post menopausal or otherwise hormonally deficient women, who have frequent need of relief of one or more symptoms of vaginal atrophy as those described above. Topical administration of the composition according to the present invention is non-invasive and can be repetitively provided.

In an embodiment of the present invention, the effective amount of the glycerophosphate salt is administered in combination with estrogen. The use of glycerophosphate salt may lower the need for estrogen, thus lower the potential risk of carcinogenesis, heart attack and stroke associated with the use of estrogen.

In yet another embodiment of the present invention, the urogenital disorder is a rectal disorder. The effective amount of a glycerophosphate salt can be administered to the rectum of the subject.

In another embodiment of the present invention, CGP can also be used in penile prosthesis. At the time of penile prosthesis insertion for erectile dysfunction, Hagar dilators are utilized to destroy and dilate the interior of the corpora cavernosum. This has the potential, in some patients, to cause trauma and scarring of the corporal capsule. That scarring could prevent the optimal function of the inflatable penile prosthesis, preventing completion of its anticipated function during sexual activity. Topical administration of CGP to the interior of the corpora cavernosum, for example, by coating the Hagar Dilators or other instruments such as dilamezinsert instruments (Lone Star Medical Products, Inc., Houston) or Rossello-Carrion dilators with CGP before inserting them for corporeal preparation, can prevent or reduce the formation of scarring resulting from penile prosthesis insertion.

In another embodiment of the present invention, CGP can also be used in percutaneous nephrostomy, a treatment modality that allows direct access to the renal pelvis via the abdominal flank by placing a guidewire into the urological collecting system over which Amplatz Dilators or inflatable dilators will allow for an access port to then be placed into the renal pelvis, allowing a nephroscope to be utilized to treat renal pelvic calculi. Scar tissue formation along that tract would complicate future intervention and with the assumption that there are limited optimal approaches to the kidney it would therefore put the patient at an increased failure of future procedures. Topical administration of CGP to the tract, for example, by coating the guidewire, the dilators, and/or the drainage catheters with CGP before their insertion can prevent or reduce the formation of scar tissue along the tract in percutaneous nephrostomy.

In yet another embodiment of the present invention, CGP can also be used in laparoscopic surgery. Laparoscope and surgical instruments are inserted through laparoscopic ports, which are traumatic dilation of access to the peritoneal cavity. Patients are at risk for scar formation, because of the traumatic dilation. Topical administration of CGP to the laparoscopic ports or coating of the laparoscope and surgical instruments with CGP would enhance healing and prevent scar formation, as has been shown by laparoscopic cutaneous studies described in Example 2 below.

Compositions useful in the present invention can be formulated using any method known to those skilled in the art in view of the present disclosure. To prepare the compositions for administration to a subject, one or more glycerophosphate salts, optionally other active ingredients, are intimately admixed with an acceptable carrier according to conventional pharmaceutical compounding techniques. The composition can contain about 0.1 mg to about 150 mg of a glycerophosphate salt per gram of the composition, and can be constituted into any form suitable for the mode of administration selected.

Carriers include one or more excipients such as binders, suspending agents, lubricants, flavorants, sweeteners, preservatives, dyes, and coatings. Carriers can take a wide variety of forms depending on the form of preparation desired for administration. For parenterals, the carriers usually comprise sterile water, though other ingredients, for example, for purposes such as aiding solubility or for preservation, can also be included. For injectable suspensions, appropriate liquid carriers, suspending agents and the like can be employed. For liquid oral preparations, for example, suspensions, elixirs and solutions, suitable carriers and additives include water, glycols, oils, alcohols, flavoring agents, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For topical compositions, such as cream or gel, suitable carriers and additives include, for example, thickeners, humectants, keratolytics, oils, emollients, surfactants, preservatives, colorants, antioxidants, perfumes, mineral oil, liquid petrolatum, white petrolatum, glycerin, polyethylene glycol and propylene glycol.

The compositions for administration herein can contain, per dosage unit, e.g., tablet, capsule, powder, injection, teaspoonful and the like, an amount of the active ingredient necessary to deliver an effective amount as described above. A glycerophosphate salt can be formulated in an extended release form suitable for once-weekly or once-monthly administration. Methods are known to those skilled in the art to manufacture the extended release dosage form.

Compositions of the present invention can be formulated at various pH levels would be suitable for purposes of the invention in view of the present disclosure. In preferred embodiments, compositions useful for the present invention have a pH of about 4.4 to about 8.6, more preferably, at a pH of about 5.45 to about 7.4.

Compositions for administration herein can be administered in many forms, such as solutions, suspensions, tablets, pills, capsules, spray, gels, drops, sustained release formulations, powders or active ingredient impregnated bandages.

In a particular embodiment of the invention, creams, gels, ointments, powders, aerosols and solutions are suitable for direct topical administration of compositions herein to the urethra of a subject. Preferably, the compositions are dermatologically acceptable and do not cause significant skin irritation under normal usage circumstances with typical patients when the compositions are applied to the skin. Compositions containing a glycerophosphate salt suitable for topical administration have been described, for example, in US2004037766 and U.S. Pat. No. 5,972,321, which are incorporated herein by references.

CGP has a relatively low water solubility. A saturated CGP solution only contains about 1% CGP. Thus, CGP exists in a suspension in a liquid composition containing higher concentrations of CGP. In the absence of a stabilizer, CGP possibly exists in gritty grains, which can be abrasive and thus irritate, not soothe, the urethral surface.

Thus, in one embodiment of the present invention, a glycerophosphate salt, such as calcium glycerophosphate, is formulated in a composition for topical application, such as a cream, gel or ointment, with a stabilizer that is inherently “slippery” as a suspending agent. Exemplary “slippery” stabilizers include, but are not limited to, a sodium carboxymethylcellulose gum, cellulose gum, agar, guar gum, carrageenan or others. When utilized at the levels of 0.25% to 5% (w/w), preferably 0.5% to 3% (w/w), the stabilizer can produce a desirably consistency of the product for such topical application.

The inclusion of one or more inherently slippery stabilizers in a CGP composition facilitates coating each and every particle of the otherwise gritty CGP, so that it does not irritate obviously already highly sensitized tissue. The slippery stabilizers also lubricate the insertion of an applicator coated with the CGP composition into sequestered areas of the urethra. The stabilizer is fully permeated with CGP. However, the stabilizer does not interfere with direct molecular contact of CGP with the epithelial skin cells. Therefore, the CGP is in effective cellular contact at all times. Upon insertion of the applicator coated with the CGP composition into the urethra, CGP extant in the combination solution/suspension is then topically self-administered to the surface of the urethra over a period of time.

In an embodiment for topical administration, the composition for administration comprises one or more glycerophosphate salts, a solvent, and at least one excipient selected from thickeners, humectants, keratolytics, oils, emollients, surfactants, preservatives, colorants, antioxidants, perfumes, mineral oil, liquid petrolatum, white petrolatum, glycerin, polyethylene glycol and propylene glycol. A thickener can be any agent useful as an aid to thicken or add structure to a topical formulation, or to result in a viscosity suitable for dermatologic applications. Non-limiting examples of thickening agents are gums and natural polysaccharides, mineral thickeners, oils, and synthetic polymeric thickeners.

In another embodiment for topical administration, the composition for administration comprises one or more solvents in an amount of about 10% to about 90% by weight, one or more glycerophosphate salts in a total amount of about 0.001% to about 25% by weight, a polymeric thickener in an amount of about 0.05% to about 5% by weight, and one or more keratolytic agents are present in a total keratolytic agent concentration amount of about 0.015% to about 25% by weight. The solvent is preferably nonalcoholic. The polymeric thickener can be a polyacrylic acid thickener or an alkylhydroxycellulose thickener. The keratolytic agent can be selected from the group consisting of alpha- and beta-hydroxycarboxylic, beta-ketocarboxylic acids, a salicylate, and salts, amides or esters thereof.

In yet another embodiment for topical administration, the composition for administration further contains a preservative. Preferably the preservative is food grade or pharmaceutical grade. Examples of preservatives that can be used in the composition include, but are not limited to, methylparaben, ethylparaben, butylparaben, propylparaben, and any other preservative that is typically used in water-based cosmetics, such as creams and lotions and some bath products. The preservative is present at an amount that is sufficient to prevent the composition from supporting the growth of microbes such as bacteria, fungi, or yeasts.

In one embodiment of the present invention, the composition can comprise one or more other agents that are useful for treating a urogenital disorder, such as those discussed above.

Another general aspect of the present invention relates to a method of diagnosing a urogenital disorder in a subject. The method comprises (1) associating an effective amount of a glycerophosphate salt with an endoscope; (2) inserting the endoscope associated with the glycerophosphate salt into the urogenital system of the subject; and (3) detecting the urogenital disorder using the endoscope.

Endoscopy is a diagnostic medical procedure that is used to assess the interior surfaces of an organ by inserting an endoscope into the body. The endoscope can be a rigid or flexible tube. It can not only provide an image for visual inspection and photography of the internal organs, but also enable taking biopsies and retrieval of foreign objects.

Coating the endoscope with a cream, gel or lotion comprising an effective amount of a glycerophosphate salt prior to its insertion into the urogenital system, not only facilitates the insertion without pain, but also hastens the healing of wounds the endoscope may cause to the delicate urogenital surface.

In another general aspect, the present invention relates to a device for treating a urogenital disorder in a subject. The device comprises an applicator associated with an effective amount of a glycerophosphate salt, and the applicator is adapted to be applied to an organ of the urogenital system of the subject, such as the urethra, vagina or rectum. In an embodiment of the present invention, the applicator is a solid rod, a catheter, a stent, a dilator, a suppository, or a sound. In another embodiment, the glycerophosphate salt is calcium glycerophosphate.

In an embodiment of the present invention, the applicator is coated or impregnated with an effective amount of a glycerophosphate salt and one or more additional active ingredients that are effective in treating urogenital disorders, such as an anti-microbial agent, an antiseptic, an immediate local anesthetic, or a longer term analgesic.

The applicators can be made by methods known to those skilled in the art in view of the present disclosure. Alternatively, the applicator can be purchased from commercial sources. Any of the solid rods, catheters, stents, dilators, suppositories, or sounds used in conventional methods for treating urogenital disorders, can be used as an applicator in the present invention.

The applicator is designed to be inserted into the organ of treatment, such as the urethra, the vagina or the rectum. The shape of the applicator can vary in sizes and shape, depending on the size and shape of the organ. For examples, in males the urethra's outer part is within the highly flexible pendulus of the penis and can be bent or straightened as necessary. The urethra's inner part extends around the pubic articulation and is therefore curved. Thus, the shape of an applicator to be used for insertion into a male urethra can generally comprise a straight portion extending from the handle, followed by a curved portion adjacent the tip. An applicator to be used for insertion into a female urethra can be much shorter than those used for male subjects.

In one embodiment of the present invention, the applicator is a solid rod. Any of the bougies used in the non-surgical dilation of urethra strictures can be used as an applicator in the present invention, such as those described in “Urology”, 3d ed., v.1, p. 242, M. F. Campbell and J. H. Harrison, eds. (Saunders, 1970). The bougies can be solid metal or plastic rods that are shaped to accommodate the physiological curvature of the urethra. The bougies can also be made of other materials and shapes that are known to those skilled in the art. Bougies with increasing size have been commonly used in the conventional treatment of urethral strictures. According to an embodiment of the present invention, an effective amount of a glycerophosphate salt is coated on all or portions of the surface of a bougy, and the drug coated bougy is then inserted into the urethra to provide improved treatment of urethral strictures.

In another embodiment, the applicator is a hollow instrument, e.g., a urinary catheter or a urethral stent, that can be inserted into the urethra. The tube allows drainage or injection of fluids or access by surgical instruments while applying an effective amount of a glycerophosphate salt to the urethra.

The process of inserting a catheter is catheterization. A catheter can be a thin, flexible tube, i.e., a “soft” catheter, or a larger, solid tube, i.e., a “hard” catheter. Depending on the use, catheters can have various sizes. They can be made of a variety of materials, such as latex, silicone, PVC, or Teflon, and types.

In a particular embodiment, the applicator is a Foley catheter, a flexible tube that is passed through the urethra during urinary catheterization and into the bladder to drain urine. It is retained by means of a balloon at the tip which is inflated with sterile water. A Foley catheter is commonly made in silicone rubber or natural rubber. However, other suitable materials can also be used. The relative size of a Foley catheter is described using French units (F). The most common sizes are 10 F to 28 F. 1 F is equivalent to 0.33 mm=0.013″= 1/77″ of diameter, or 30 French units=1 cm. However, other sizes can also be used. Any sub-types of Foley catheters can be used as an applicator in the present invention, such as a Coude (French for elbowed) catheter, which has a 45° bend at the tip to allow easier passage through an enlarged prostate; or a council tip catheter, which has a small hole at the tip that allows it to be passed over a wire. Foley catheters can also be used as an applicator in a female subject. The catheter can be inserted behind the cervical wall and inflated.

The effective amount of a glycerophosphate salt can be administered to the urethra by first coating it on the surface of a catheter and then inserting the drug-coated catheter into the urethra. Alternatively, it can also be administered to the urethra by injection through a catheter that has already been inserted in the urethra, for example, via a pressure, gravity, infusion device, etc.

In another particular embodiment, the applicator is a urethral stent, a completely internal device that is used to keep open the urethra and allow the passing of urine. For example, the applicator can be a temporary prostatic stent that can be inserted into and removed from the urethra in a similar manner as that of a Foley catheter, requiring only topical anesthesia. The temporary prostatic stent can be coated or impregnated with the effective amount of a glycerophosphate salt before it is inserted into the urethra. In another example, the applicator can be a council tip catheter that is lubricated/coated with a cream containing an effective amount of CGP in advance of passage of the catheter to the urethra.

In another embodiment of the present invention, the applicator is a dilator, which is a surgical instrument or medical implement used to induce dilation. It can be used to expand an opening or passage such as the cervix, urethra, or vagina in the urogenital system. In an embodiment of the present invention, the applicator is a bougie a boule dilator used for the female urethra.

In another embodiment of the present invention, the applicator is a suppository, which is a drug delivery system that is inserted either into the rectum (rectal suppository), vagina (vaginal suppository) or urethra (urethral suppository) where it dissolves. Vaginal suppositories are commonly used to treat gynecological ailments, including vaginal infections such as candidiasis. Rectal suppositories are commonly used for laxative purposes, for example, with chemicals such as glycerin or bisacodyl. Rectal suppositories are also used to treat a hemorrhoid by delivering a moisturizer or vasoconstrictor, for delivery of a systemically-acting medication, such as promethazine or aspirin, or a general substance, such as paracetamol (acetaminophen), diclofenac, opiates, and eucalyptol crosses the rectal mucosa into the bloodstream.

The suppositories can be made from a greasy base, such as cocoa butter, in which the glycerophosphate salt and other excipients are dissolved. The grease will melt at body temperature. The suppositories can also be made from a water soluble base, such as polyethylene glycol. Suppositories made from polyethylene glycol are commonly used in vaginal and urethral suppositories.

Any of the suppositories described above or known to those skilled in the art can be used to deliver an effective amount of a glycerophosphate salt into an organ of the urogenital system in view of the present disclosure.

In another embodiment of the present invention, the applicator is a sound, which is an instrument for probing and dilating a passage within the body. The sound can be used to increase the inner diameter of the body passage and to locate obstructions in it.

In one embodiment, the sound is a urethral sound that is designed to be inserted into the male or female urethra, for the purpose of stretching or unblocking a stricture. Examples of urethral sounds that can be used in the present invention include, but are not limited to Bakes sounds, also known as rosebud or bullet sounds, which have a long thin metal rod with a bulbous bud on the end; Dittel sounds, which have a flat end and a rounded end; Henk sounds, which have a more pronounced curve at the ends, as well as a metal rib on each end; Pratt sounds, which are longer urethral dilators (double ended ones are usually almost a foot long) with rounded and slightly bent ends; and Van Buren sounds, which have a pronounced curve at each end, specifically for the purpose of reaching the bladder.

In another embodiment, the sound is a uterine sound, that is intended for probing a woman's uterus through the cervix, to determine the level of dilation or to induce further dilation. Examples of uterine sounds that can be used in the present invention include, but are not limited to, Hegar dilators, which have two rounded ends, are fairly short, and are mildly curved in shape; and Sims sounds, which have a flat end and a rounded end.

In another embodiment of the present invention, the applicator is a vaginal applicator, such as those that have been used for topical application of estrogen to the vagina, e.g., suppositories, vaginal stent, vaginal catheter, vaginal ring, etc.

In another embodiment of the present invention, the applicator is a rectal applicator, such as rectal suppositories, rectal thermometers, etc.

In yet another embodiment, the applicator is an endoscope that is adapted for insertion into an organ of the urogenital system.

The applicator can be coated or impregnated with the effective amount of a glycerophosphate salt using methods known to those skilled in the art in view of the present disclosure.

In one embodiment, a composition comprising the effective amount of a glycerophosphate salt, such as a CGP lotion, is applied to an applicator by dipping the applicator directly in the composition.

In another embodiment, the glycerophosphate salt composition and one or more coating polymers can be mixed and/or dissolved in a common solvent or a pair of solvents. The glycerophosphate salt-polymer mixture is sprayed or dipped on an applicator, forming a drug polymer matrix on the surface of the applicator upon drying. By controlling the compositions of the drug polymer matrix, the glycerophosphate salt can be released from the coated applicator in a time-release manner, e.g., releasing from the applicator over hours, weeks or even months.

A smooth surface of the applicator is desirable because applicators with a rough surface may result in more cell adhesion, inflammation, and strictures than a smoothly polished stent. An adhesive layer or primer that is biocompatible, is used to promote good adhesion between the drug polymer matrix and the surface of an applicator. For example, a phenoxy primer coating can be used to increase the adhesiveness of a drug polymer matrix comprising an effective amount of glycerophosphate salt to a smooth surface of a metallic or polymeric based applicator.

In one embodiment of the present invention, the applicator is additionally coated with one or more agents, such as silver alloy, to reduce infections, upon insertion of the applicator into the urethra.

Another general aspect of the present invention relates to a kit for treating a urogenital disorder in a subject. The kit comprises a composition comprising an effective amount of a glycerophosphate salt; and instructions for administering the composition to an organ of the urogenital system of the subject.

Any of the compositions that can be used in methods according to embodiments of the present invention can be included in the kit. The composition can comprise a glycerophosphate salt as the only active ingredient. The composition can also comprise a combination of a glycerophosphate salt and one or more additional active ingredients selected from the group consisting of an antiseptic, an immediate local anesthetic, and a longer term analgesic.

The kit can further comprise an applicator, such as a solid rod, a catheter, a stent, a dilator, a suppository, or a sound, for application of the composition to the an organ of the urogenital system, such as the urethra, vagina or rectum, of the subject. The applicator can be pre-coated with an effective amount of the glycerophosphate salt composition. Alternatively, the applicator can be free of the glycerophosphate salt composition, but will be coated or otherwise associated with the composition immediately prior to the application of the applicator to the urogenital system.

In one embodiment of the present invention, the kit comprises a composition comprising an effective amount of a glycerophosphate salt; and instructions for administering the composition to the urethra of the subject for treating a urethral disorder, such as urethritis, urethral abscess, urethral stricture, urethral fistula, urethral diverticulum, urethral trauma, sympathetic inflammation or urethral caruncle. In a preferred embodiment, the kit further comprises an applicator for the application of the composition into the urethra.

In another embodiment of the present invention, the kit comprises a composition comprising an effective amount of a glycerophosphate salt; and instructions for administering the composition to the vulva, the vagina, the cervix, or the outer urinary tract of the subject for treating a genital disorder, such as vaginitis or vaginal atrophy, surgical and trauma wounds, etc. In a preferred embodiment, the kit further comprises an applicator for the application of the composition into the vulva, the vagina, the cervix, or the outer urinary tract.

In yet another embodiment of the present invention, the kit comprises a composition comprising an effective amount of a glycerophosphate salt; and instructions for administering the composition to the rectum of the subject for treating a rectal disorder, such as proctalgia or rectal prolapse, hemorrhoids, radiation proctitis, rectal fissure, and rectal stricture. In a preferred embodiment, the kit further comprises an applicator for the application of the composition into the rectum.

This invention will be better understood by reference to the non-limiting example that follows, but those skilled in the art will readily appreciate that the example is only illustrative of the invention.

### Example 1

### Treatment of Urethral Strictures

AkPharma's Cellerity® 7.5 topical lotion comprises the following ingredients:

| Ingredients | Concentration (w/w) |
|---|---|
| Water | 88.00%* |
| Calcium glycerophosphate | 7.50% |
| Cellulose gum | 2.75% |
| DL-lactic acid | 2.00%* +/− 1% to bring pH to about 5.65 |
| Glycerin | 1.00% |
| Methyl paraben | 0.20% |

Due to variations in pH, the amount of water or lactic acid used in the lotion may vary slightly, so the above representative formulation does not add up to exactly 100%.

As described below, Cellerity® 7.5 has been used for treating different types of urethral strictures in various patients.

Patient No. 1 suffered from a long standing fossa navcularis stricture (distal part of penile urethra). The stricture was initially dilated with urological sounds. The patient was instructed to self-dilate the distal 1½″ with a rectal thermometer coated with Cellerity® 7.5 twice a day. The patient was advised not to void for about two hours after the administration of Cellerity® 7.5. After about three weeks treatment, unexpectedly, this patient has had responded dramatically to this therapy. He has had no recurrence of the urethral stricture and had been able to maintain an adequate urethral caliber of approximately 20 FR (6.6 mm), which is within the normal range. These results were surprising, because these strictures have a natural tendency to recur rapidly.

Patient No. 2 suffered a long standing mid-urethral stricture, for which he has self-catheter dilated for over twenty years. He was instructed to void, drain his bladder, and self-catheterize with a Cellerity® 7.5 coated catheter. The patient was advised not to void for about two hours after the administration of Cellerity® 7.5. After about 4 weeks of treatment, he has reduced the need for repeated dilatations and has been able to maintain adequate urethral caliber of approximately 16-18 FR.

Patient No. 3 suffered multiple urological procedures, which caused a proximal pendulous urethral stricture, requiring progressively more frequent urethral dilatations. He was not able to self-dilate, but intermittently used Cellerity® 7.5 as a lubricant to dilatation. Although the results were not as pronounced as in patients who self-dilated at least weekly, patient No. 3 experienced less severe stricture recurrence when dilating with Cellerity® 7.5 than when dilating without Cellerity® 7.5.

Patient No. 4 had a 20-30 year history of urethral strictures in the proximal pendulous urethra, which consistently recurred every 6-12 weeks and required intervention and treatment. He underwent a cystoscopy and urethral dilatation and was treated at that time with Cellerity® 7.5 as a lubricant to dilatation. At a follow-up visit approximately four weeks after the treatment, the patient stated that the treatment was the best he had received in the last 20 years. At the visit, his urethra had minimal scar formation and he again received a treatment with Cellerity® 7.5.

Patient No. 5 had a fossa navcularis stricture. He performed weekly self-dilating with a rectal thermometer and used Cellerity® 7.5 as a lubricant to dilatation. Since initiating this treatment, he has had no recurrence of his stricture.

### Example 2

### Laparoscopic Cutaneous Studies

AkPharma's Cellerity® 10 topical lotion comprises the following ingredients:

| Ingredients | Concentration (w/w) |
|---|---|
| Water | 85.00%* |
| Calcium glycerophosphate | 10.00% |
| Cellulose gum | 2.50% |
| DL-lactic acid | 2.00%* +/− 1% to bring pH to about 5.65 |
| Glycerin | 1.00% |
| Methyl paraben | 0.20% |

Due to variations in pH, the amount of water or lactic acid used in the lotion may vary slightly, so the above representative formulation does not add up to exactly 100%.

Cellerity® 10 topical lotion has been used in single patient multiple sites laparoscopic procedure studies. The studies compared healing time, severity of scar formation and inflammation at laparoscopic ports with and without CGP treatment. The studies clearly showed that, as compared with the untreated port sites, treating laparoscopic ports with CGP has resulted in a decrease in the total healing time, a dramatic reduction in inflammatory response, and substantially less fibrosis as identified by the pliability and general consistency of the healed surgical site.

For example, the results of CGP treatment was blatantly obvious in a patient treated only one half of the lap port sites with Cellerity® 10 topical lotion. The treatment was started within 24 hours after the laparoscopic surgery. The patient was instructed to allow the applied lotion to dry completely before applying his clothes and not to wash the treatment area for two hours after the application. This procedure was performed twice a day for three full weeks. The patient had no ill effects to the course of treatment. The untreated ports were grossly inflamed, had tissue necrosis at the incision site and desquamation of epithelium. The untreated ports also remained inflamed and the staple sites were still prominent and erythematous. At the incision line, a glassy layer of fibroblastic scar tissue was present. However, at the treated port sites, there was no tissue necrosis, no desquamation, minimal inflammation and no glassy fibroblastic presence.

Applying CGP via Steri-Strips to the lap port sites did not appear to have affected the efficacy. For example, in another patient, CGP treatment was started 24 hours after laparoscopic surgery, by applying Cellerity 10 topical lotion directly to the right side of the incisions and through Steri-Strips to the left side of the incisions, and leaving the midline left untreated. At 10 days after the treatment, there was no difference between the right and left sides, indicating that Steri-Strips had no effect on the efficacy of the CGP treatment. When compared to the untreated midline incision, there was substantially less inflammation, no edematous changes and less erythema in the left and right sides treated with CGP. The untreated midline scar remained as inflamed and delayed in the healing process by 3-4 weeks, because the inflamed situation must resolve before the healing can occur. That delay appeared to have allowed more fibroblastic growth in the untreated scar, than was present on the treated scars.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of treating a urogenital disorder in a subject, comprising topically administering an effective amount of a glycerophosphate salt to an organ of the urogenital system of the subject, wherein the organ suffers from the urogenital disorder.

2. The method of claim 1, comprising
(a) associating the effective amount of the glycerophosphate salt with an applicator; and
(b) placing the applicator associated with the glycerophosphate salt into the organ.

3. The method of claim 2, wherein the applicator is a solid rod, a catheter, a stent, a dilator, a suppository, or a sound.

4. The method of claim 2, wherein the effective amount of the glycerophosphate salt is administered to the organ through a catheter already placed in the organ.

5. The method of claim 2, wherein the glycerophosphate salt is administered in a composition adapted for topical administration to the organ.

6. The method of claim 5, wherein the composition comprises a stabilizer selected from the group consisting of a sodium carboxymethylcellulose gum, cellulose gum, agar, guar gum, and carrageenan.

7. The method of claim 1, wherein the effective amount of glycerophosphate salt is 0.1% to 15% by weight.

8. The method of claim 1, wherein the urogenital disorder is a urethral disorder and the organ is the urethra of the subject.

9. The method of claim 8, wherein the urethral disorder is selected from the group consisting of a urethral stricture, urethritis, urethral abscess, urethral fistula, urethral diverticulum, urethral caruncle, urethral trauma, and sympathetic inflammation.

10. The method of claim 8, wherein the urethral disorder is a urethral stricture.

11. The method of claim 1, wherein the urogenital disorder is a genital disorder and the organ is the vulva, the vagina, the cervix, or the outer urinary tract of the subject.

12. The method of claim 11, wherein the genital disorder is selected from the group consisting of vaginitis, vaginal atrophy, wounds, and vaginal trauma.

13. The method of claim 11, wherein the genital disorder is vaginal atrophy.

14. The method of claim 11, further comprising administering an effective amount of estrogen to the subject.

15. The method of claim 1, wherein the urogenital disorder is a rectal disorder and the organ is the rectum of the subject.

16. The method of claim 15, wherein the rectal disorder is selected from the group consisting of proctalgia, rectal prolapse, hemorrhoids, radiation proctitis, rectal fissure, and rectal stricture.

17. The method of claim 1, further comprising a surgical treatment of the urogenital disorder in the organ, wherein the glycerophosphate salt is first administered to the organ about 0 to about 7 days after the surgical treatment.

18. The method of claim 17, wherein the surgical treatment is selected from the group consisting of penile prosthesis, percutaneous nephrostomy and laparoscopic surgery.

19. The method of claim 1, further comprising one or more treatment methods selected from the group consisting of dilation, catheterization, internal urethrotomy, urethroplasty and in-dwelling stents.

20. The method of claim 19, wherein the effective amount of the glycerophosphate salt is administered to the organ before, after, or simultaneously with the one or more other treatment methods.

21. The method of claim 1, further comprising administering to the subject a medication selected from the group consisting of clotrimazole, doxycycline, fluconazole, metronidazole, nitrofurantoin, nystatin, clindamycin, Betadine®, hexaclorophene, or a combination of sulfamethoxazole and trimethoprim.

22. The method of claim 1, wherein the glycerophosphate salt is selected from the group consisting of calcium glycerophosphate, magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, glycerophosphate strontium, glycerophosphate silver, and combinations thereof.

23. The method of claim 1, wherein the glycerophosphate salt is calcium glycerophosphate.

24. The method of claim 1, wherein the subject is a human subject.

25. A device for treating a urogenital disorder in a subject, comprising an applicator associated with an effective amount of a glycerophosphate salt, wherein the applicator is adapted for placement into an organ of the urogenital system of the subject.

26. The device of claim 25, wherein the applicator is a solid rod, a catheter, a stent, a dilator, a suppository, or a sound.

27. The device of claim 25, wherein the applicator is adapted for placement into the urethra, vulva, vagina, cervix, outer urinary tract, or rectum.

28. The device of claim 25, wherein the applicator is coated with the effective amount of a glycerophosphate salt.

29. The device of claim 25, wherein the applicator is impregnated with the effective amount of a glycerophosphate salt.

30. A kit for treating a urogenital disorder in a subject, comprising:
(1) a composition comprising an effective amount of a glycerophosphate salt; and
(2) instructions for administering the composition to an organ of the urogenital system of the subject.

31. The kit of claim 30, wherein the glycerophosphate salt is calcium glycerophosphate.

32. The kit of claim 30, further comprising an applicator for application of the composition into the organ.

33. The kit of claim 30, wherein the urogenital disorder is a urethral stricture and the organ is the urethra.

34. The kit of claim 30, wherein the urogenital disorder is vaginal atrophy and the organ is vulva, vagina, cervix, or outer urinary tract of the subject.

35. A method of diagnosing a urogenital disorder in a subject, comprising:
a. associating an effective amount of a glycerophosphate salt with an endoscope;
b. inserting the endoscope associated with the glycerophosphate salt into the urogenital system of the subject; and
c. detecting the urogenital disorder using the endoscope.
